(19) United States
(12) Patent Application Publication
(10) Pub. No.: US 2011/0000008 A1
(43) Pub. Date: Jan. 6, 2011

(54) Goggles strap with case

(75) Inventor: Chih-Cheng Shiue, Escondido, CA (US)

Correspondence Address: Guice Patents PLLC, 12647 Galveston Court #302, Manassas, VA, 20112, US

(73) Assignee: QDS Injection Molding LLC.

(21) Appl. No.: 11/826,821

(22) Filed: Jul. 18, 2007

(30) Foreign Application Priority Data
May 28, 2007 (TW) 96208676

(51) Int. Cl. A61F 9/02

(52) U.S. Cl. 2/452

(57) ABSTRACT

In one embodiment a head strap of goggles or mask includes a case at about an intermediate position of the strap and having a hole at either side. A first cord securely passes a first channel in one section of the strap to have one end being exposed to form a first speaker and the other end being disposed in the case by passing one hole. A second cord securely passes a second channel in the other section of the strap to have one end being exposed to form a second speaker and the other end being disposed in the case by passing the other hole. A music player (e.g., MP3 audio player) is adapted to store in the case and is operatively connected to a plug at a joining portion of the other ends of the first and second cords in the case. The case and/or the music player can be waterproof.

### BACKGROUND OF THE INVENTION

1. Field of Invention

The invention relates to strap of goggles or mask and more particularly to such a strap having a case for storing a music player (e.g., MP3 audio player).

2. Description of Related Art

Conventionally, it is very difficult of mounting a waterproof music player on the head while engaging in water sports. While a variety of lightweight music players are available, it is still not possible of satisfactorily mounting it on a piece of water sports equipment.

For example, there is a type of MP3 audio player for water sports available. Such MP3 audio player is either waterproof or is permanently stored in a waterproof case as a part of goggles or mask. However, its price is very high. Thus, market acceptance is very low. Moreover, it is disadvantageous due to large size, complex circuitry, and high cost of maintenance. Thus, the need for improvement still exists.

### SUMMARY OF THE INVENTION

It is therefore one object of the invention to provide goggles or mask having a case on the strap for storing a music player (e.g., MP3 audio player) in which either the case is waterproof or the music player is waterproof.

In one aspect of the invention cords, a plug, and speakers are mounted in the strap and the MP3 audio player is mounted in the case to be operatively connected to the plug.

In another aspect of the invention the case is releasably secured to the strap.

In a further aspect of the invention the cords, the plug, and the speakers are waterproof.

The above and other objects, features and advantages of the invention will become apparent from the following detailed description taken with the accompanying drawings.

### BRIEF DESCRIPTION OF THE DRAWINGS

FIG. 1 is an environmental view of swimming goggles incorporating a waterproof case on the strap for storing a music player in accordance with the invention;

FIG. 2 is a perspective view of the strap of FIG. 1 with the case being open;

FIG. 3 is a view similar to FIG. 2 where a music player, cords, a plug, and speakers are to be mounted in the strap and the case;

FIG. 4 is a perspective view of another embodiment of goggles strap according to the invention;

FIG. 5 is a sectional view of the case of FIG. 4;

FIGS. 6, 7 and 8 are sectional views showing the fastening of cover and body of the case in three different configurations respectively;

FIG. 9 is an exploded perspective view of the protective pad and the speaker; and

FIG. 10 is an exploded perspective view of another embodiment of the case according to the invention.

### DETAILED DESCRIPTION OF THE INVENTION

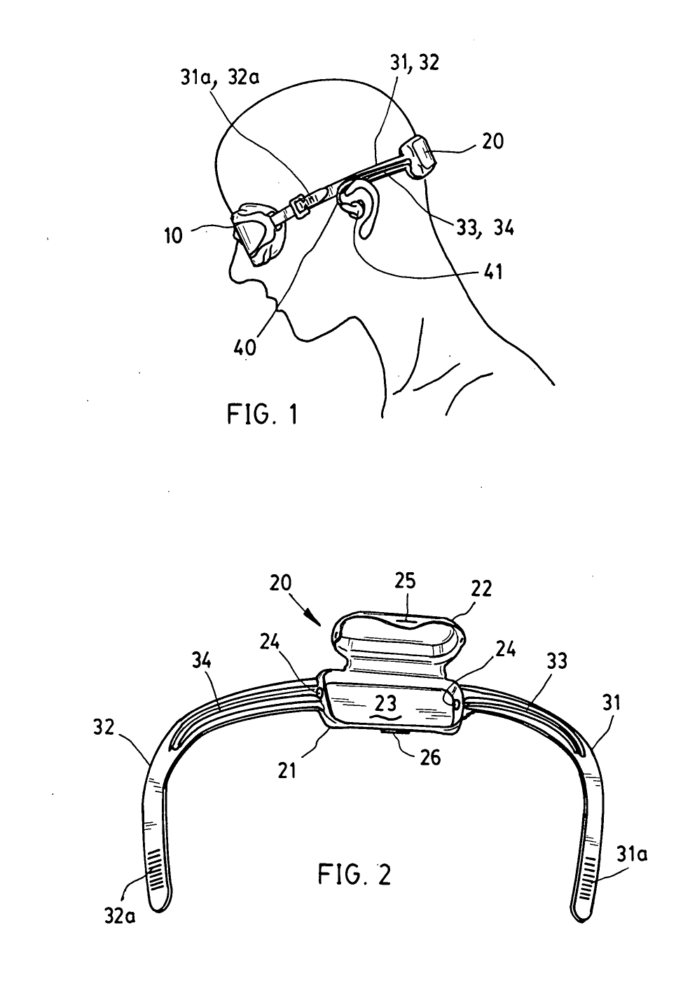

Referring to FIGS. 1 to 3, swimming goggles 10 having a waterproof case on the strap in accordance with the invention is shown. Note that the swimming goggle is only an exemplary example, it is possible of mounting the waterproof case on the strap of a swim mask, safety mask, or the like in other embodiments without departing from the scope of the invention.

The strap is a conventional one having two ends 31a and 32a adapted to secure to endpieces of an eye frame of the goggles 10. Also, a user can adjust the tightness of the strap in a manner known in the art.

The strap comprises left and right strap sections 31, 32 each having an end 31a or 32a, and an openable waterproof case 20 formed in a portion (e.g., intermediate portion) interconnecting the strap sections 31, 32.

The strap is flexile in nature. Also, the case 20 is formed of a thermoplastic material. The case 20 is rectangular and comprises a hollow body 21 with a receiving space 21 defined therein, the body 21 having two side holes 24, and a cover 22 flexibly formed with the body 21 and adapted to open or close. Moreover, the cover 22 has a tab 25 on one edge and the body 21 has a slit 26 on one edge. The tab 25 is adapted to insert into the slit 26 for closing the case 20 and making the case 20 waterproof.

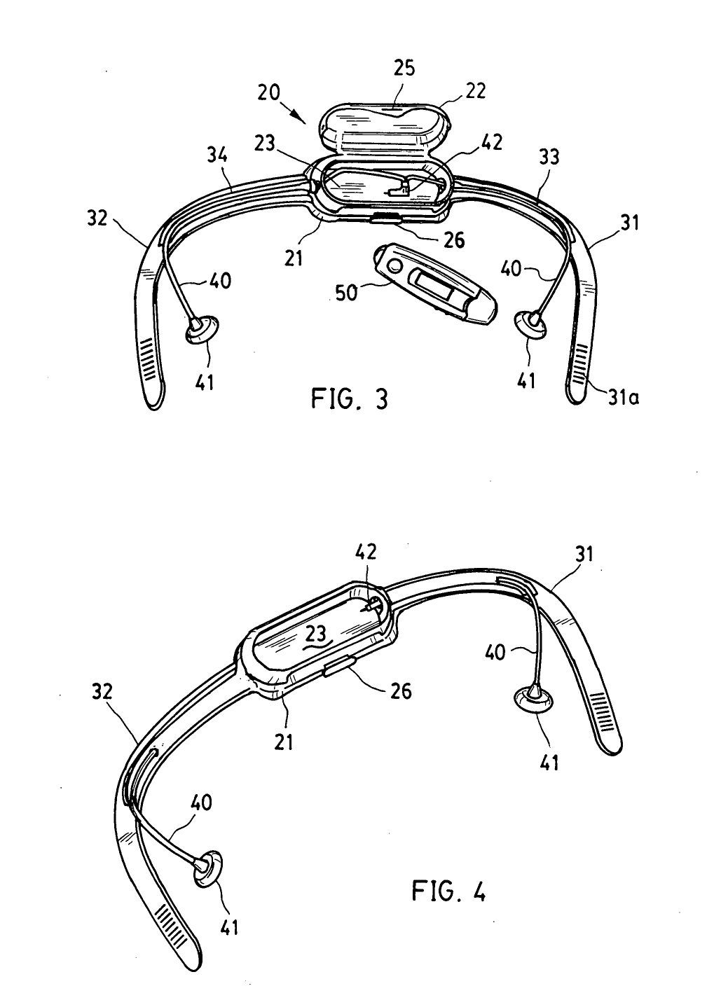

The strap sections 31, 32 are formed with channels 33, 34 respectively. Each of the channels 33, 34 starts from the hole 24 and terminates at an opening (not shown) on the strap section. As shown in FIG. 3, one cord 40 is fastened in the channel 33 and has a right speaker 41 at one end extending out of the strap section 31 and the other cord 40 is fastened in the channel 34 and has a left speaker 41 at one end extending out of the strap section 31 respectively. A plug 42 is disposed in the space 21 and is at a joining portion of the other ends of the cords 40. A user cannot move the cords 40 by pulling since the cords 40 are fixed in the channels 33, 34 after being manufactured in, for example, an injection molding process. Moreover, the speakers 41, the cords 40, and the plug 42 are made waterproof. In use, a user may simply insert the plug 42 into a jack of a music player (e.g., MP3 audio player) 50.

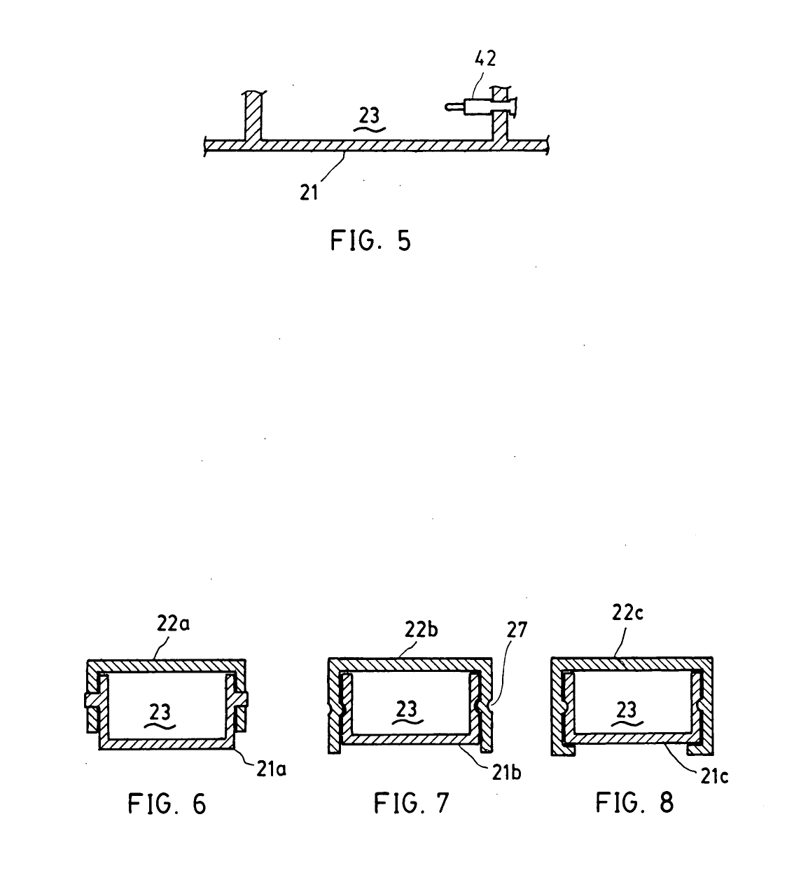

Referring to FIGS. 4 and 5, another embodiment of goggles strap according to the invention is shown in which one hole is formed at one side of the body 21, a portion of one cord 40 is formed within and along the other edge of the body 21, and the plug 42 at a joining portion of the cords 40 is secured at the hole and projects into the space 23.

Referring to FIGS. 6, 7 and 8, the cover 22 and the body 21 can be secured together by one of three methods detailed below. In FIG. 6, the cover 22a has through holes on both sides, the body 21a has projections on both sides, and the projections are adapted to tightly insert into the through holes for forming a waterproof case. In FIG. 7, the cover 22b has a raised member (and an aligned recessed member) on either side, the body 21b has a groove on either side, the raised members are adapted to tightly insert in the grooves for forming a waterproof case. In FIG. 8, the cover 22c has a raised member on either side, the body 21c has a groove on either side, the raised members are adapted to tightly insert in the grooves for forming a waterproof case.

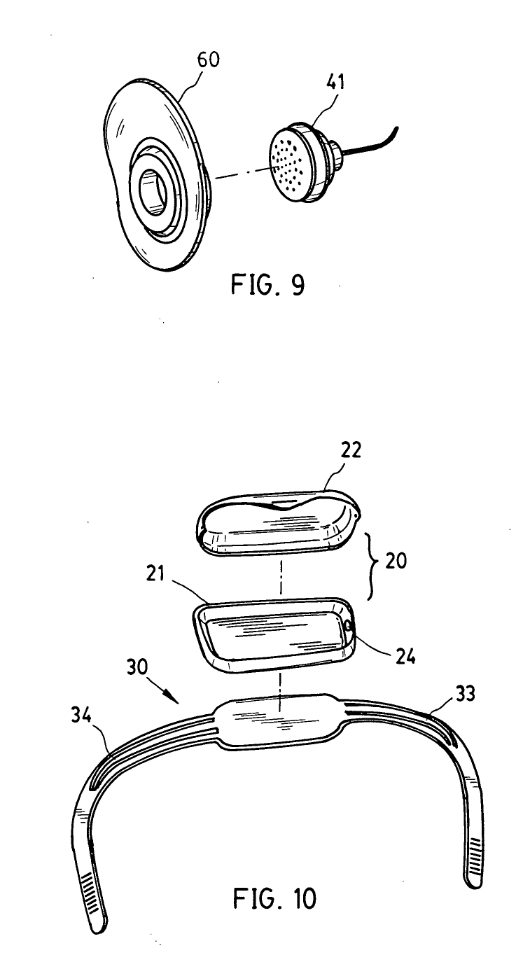

Referring to FIG. 9, a protective pad 60 is adapted to secure onto either speaker 41 for making the speaker 41 waterproof.

Referring to FIG. 10, another embodiment of the case according to the invention is shown. The strap 30 and the case 20 are manufactured separately prior to securing together by applying adhesive, snapping, screwing, or the like. One hole 24 is formed at one side of the body 21 and optionally the other hole (not shown) is formed at the other side of the body 21. Moreover, the body 21 and the cover 22 can be manufactured separately prior to securing together by applying adhesive, snapping, screwing, or the like.

Note that the case can be non-waterproof as long as, for example, the music player (e.g., MP3 audio player) is waterproof in other embodiments.

In brief, it is envisaged by the invention that, for example, a swimmer may wear the inventive strap to listen to music by operating the music player in the case while engaging in water sports.

While the invention herein disclosed has been described by means of specific embodiments, numerous modifications and variations could be made thereto by those skilled in the art without departing from the scope and spirit of the invention set forth in the claims.

What is claimed is:

1. A strap for holding to the head by adjustably securing both ends to both ends of a frame of a goggles or a mask, comprising:
an openable case at a predetermined position of the strap and having two holes at both sides respectively;
wherein a first cord securely passes a first channel in one section of the strap to have one end being exposed to form a first speaker and the other end being disposed in the case by passing one hole;
wherein a second cord securely passes a second channel in the other section of the strap to have one end being exposed to form a second speaker and the other end being disposed in the case by passing the other hole;
wherein a plug is formed at a joining portion of the other ends of the first and the second cords in the case; and
wherein a music player is adapted to store in the case and is operatively connected to the plug.

2. The strap of claim 1, wherein the predetermined position is at about an intermediate position of the strap.

3. The strap of claim 1, wherein the case and the strap are formed integrally.

4. The strap of claim 1, wherein the case and the strap are formed separately prior to securing together.

5. The strap of claim 1, wherein the case is waterproof.

6. The strap of claim 1, wherein the music player is a waterproof MP3 audio player.

7. A strap for holding to the head by adjustably securing both ends to both ends of a frame of a goggles or a mask, comprising:
an openable case at a predetermined position of the strap and having a hole at one side;
wherein a first cord securely passes a portion of the case and a first channel in one section of the strap to have one end being exposed to form a first speaker and the other end being disposed at the hole;
wherein a second cord securely passes a second channel in the other section of the strap to have one end being exposed to form a second speaker and the other end being disposed at the hole;
wherein a plug is formed at a joining portion of the other ends of the first and the second cords at the hole; and
wherein a music player is adapted to store in the case and is operatively connected to the plug.

8. The strap of claim 7, wherein the predetermined position is at about an intermediate position of the strap.

9. The strap of claim 7, wherein the case and the strap are formed integrally.

10. The strap of claim 7, wherein the case and the strap are formed separately prior to securing together.

11. The strap of claim 7, wherein the case is waterproof.

12. The strap of claim 7, wherein the music player is a waterproof MP3 audio player.
